SEQUENCE LISTING (32 sequences)

SEQ ID NO: 1 - protein, 10 aa, synthetic construct
GFTFSNYSMS

SEQ ID NO: 2 - protein, 17 aa, synthetic construct
GIYYGSGNKYYADSVKG

SEQ ID NO: 3 - protein, 7 aa, synthetic construct
NLASFDY

SEQ ID NO: 4 - protein, 13 aa, synthetic construct
SGSSSNIGSNSVN

SEQ ID NO: 5 - protein, 7 aa, synthetic construct
SNSHRPS

SEQ ID NO: 6 - protein, 9 aa, synthetic construct
GTWDSSLSG

SEQ ID NO: 7 - protein, 116 aa, synthetic construct
EVQLLESGGGLVQPGGSLRLSCAASGFTFSNYSMSWVRQAPGKGLEWVSGIYYGSGNKYYADSVKGRFTISRDNSKNTLYLQMNSLRAEDTAVYYCAKNLASFDYWGQGTLVTVSS

SEQ ID NO: 8 - protein, 110 aa, synthetic construct
QSVLTQPPSASGTPGQRVTISCSGSSSNIGSNSVNWYQQLPGTAPKLLIYSNSHRPSGVPDRFSGSKSGTSASLAISGLRSEDEADYYCGTWDSSLSGYVFGGGTKLTVL

SEQ ID NO: 9 - protein, 10 aa, synthetic construct
GFTFSGYAMS

SEQ ID NO: 10 - protein, 17 aa, synthetic construct
AISHGGSSKYYADSVKG

SEQ ID NO: 11 - protein, 18 aa, synthetic construct
DLLSPLQSIGSYDDAMDV

SEQ ID NO: 12 - protein, 13 aa, synthetic construct
SGSSSNIGSNTVS

SEQ ID NO: 13 - protein, 7 aa, synthetic construct
ADNNRPS

SEQ ID NO: 14 - protein, 9 aa, synthetic construct
ASWDDSLNA

SEQ ID NO: 15 - protein, 127 aa, synthetic construct
EVQLLESGGGLVQPGGSLRLSCAASGFTFSGYAMSWVRQAPGKGLEWVSAISHGGSSKYYADSVKGRFTISRDNSKNTLYLQMNSLRAEDTAVYYCAKDLLSPLQSIGSYDDAMDVWGQGTLVTVSS

SEQ ID NO: 16 - protein, 110 aa, synthetic construct
QSVLTQPPSASGTPGQRVTISCSGSSSNIGSNTVSWYQQLPGTAPKLLIYADNNRPSGVPDRFSGSKSGTSASLAISGLRSEDEADYYCASWDDSLNAYVFGGGTKLTVL

SEQ ID NO: 17 - DNA, 30 bp, synthetic construct
  source 1..30: mol_type = other DNA
ggattcacctttagcaattattctatgagc

SEQ ID NO: 18 - DNA, 51 bp, synthetic construct
  source 1..51: mol_type = other DNA
gggatctattatggtagtggtaataaatattacgctgattctgtaaaaggt

SEQ ID NO: 19 - DNA, 21 bp, synthetic construct
  source 1..21: mol_type = other DNA
aatctggcttcgttcgactac

SEQ ID NO: 20 - DNA, 39 bp, synthetic construct
  source 1..39: mol_type = other DNA
agtggctcttcatctaatattggcagtaattctgtcaac

SEQ ID NO: 21 - DNA, 21 bp, synthetic construct
  source 1..21: mol_type = other DNA
tctaatagtcatcggccaagc

SEQ ID NO: 22 - DNA, 27 bp, synthetic construct
  source 1..27: mol_type = other DNA
ggtacttgggattctagcctgagtggt

SEQ ID NO: 23 - DNA, 348 bp, synthetic construct
  source 1..348: mol_type = other DNA
gaggtgcagctgttggagtctgggggaggcttggtacagcctggggggtccctgagactctcctgtgcagcctctggattcacctttagcaattattctatgagctgggtccgccaggctccagggaaggggctggagtgggtctcagggatctattatggtagtggtaataaatattacgctgattctgtaaaaggtcggttcaccatctccagagacaattccaagaacacgctgtatctgcaaatgaacagcctgagagccgaggacacggccgtgtattactgtgcgaaaaatctggcttcgttcgactactggggccagggtacactggtcaccgtgagctca

SEQ ID NO: 24 - DNA, 330 bp, synthetic construct
  source 1..330: mol_type = other DNA
cagtctgtgctgactcagccaccctcagcgtctgggacccccgggcagagggtcaccatctcttgtagtggctcttcatctaatattggcagtaattctgtcaactggtaccagcagctcccaggaacggcccccaaactcctcatctattctaatagtcatcggccaagcggggtccctgaccgattctctggctccaagtctggcacctcagcctccctggccatcagtgggctccggtccgaggacgaggctgattattactgtggtacttgggattctagcctgagtggttatgtcttcggcggaggcaccaagctgacggtccta

SEQ ID NO: 25 - DNA, 30 bp, synthetic construct
  source 1..30: mol_type = other DNA
ggattcacctttagcggttatgctatgagc

SEQ ID NO: 26 - DNA, 51 bp, synthetic construct
  source 1..51: mol_type = other DNA
gcgatctctcatggtggtagtagtaaatattacgctgattctgtaaaaggt

SEQ ID NO: 27 - DNA, 54 bp, synthetic construct
  source 1..54: mol_type = other DNA
gatcttcttagtcctctgcagagtattgggtcttatgatgatgctatggacgtc

SEQ ID NO: 28 - DNA, 39 bp, synthetic construct
  source 1..39: mol_type = other DNA
agtggctcttcatctaatattggcagtaatactgtctcc

SEQ ID NO: 29 - DNA, 21 bp, synthetic construct
  source 1..21: mol_type = other DNA
gctgataataatcggccaagc

SEQ ID NO: 30 - DNA, 27 bp, synthetic construct
  source 1..27: mol_type = other DNA
gcttcttgggatgatagcctgaatgct

SEQ ID NO: 31 - DNA, 381 bp, synthetic construct
  source 1..381: mol_type = other DNA
gaggtgcagctgttggagtctgggggaggcttggtacagcctggggggtccctgagactctcctgtgcagcctctggattcacctttagcggttatgctatgagctgggtccgccaggctccagggaaggggctggagtgggtctcagcgatctctcatggtggtagtagtaaatattacgctgattctgtaaaaggtcggttcaccatctccagagacaattccaagaacacgctgtatctgcaaatgaacagcctgagagccgaggacacggccgtgtattactgtgcgaaagatcttcttagtcctctgcagagtattgggtcttatgatgatgctatggacgtctggggccagggtacactggtcaccgtgagctca

SEQ ID NO: 32 - DNA, 330 bp, synthetic construct
  source 1..330: mol_type = other DNA
cagtctgtgctgactcagccaccctcagcgtctgggacccccgggcagagggtcaccatctcttgtagtggctcttcatctaatattggcagtaatactgtctcctggtaccagcagctcccaggaacggcccccaaactcctcatctatgctgataataatcggccaagcggggtccctgaccgattctctggctccaagtctggcacctcagcctccctggccatcagtgggctccggtccgaggatgaggctgattattactgtgcttcttgggatgatagcctgaatgcttatgtcttcggcgggggcaccaagctgacggtccta

(19) United States
(12) Patent Application Publication
(10) Pub. No.: US 2026/0097129 A1
(43) Pub. Date: Apr. 9, 2026

(54) ANTIBODY-DRUG CONJUGATE COMPRISING DRUG AND ANTIBODY SPECIFICALLY BINDING TO GRP94 OR ANTIGEN-BINDING FRAGMENT THEREOF

(71) Applicant: KOOKMIN UNIVERSITY INDUSTRY ACADEMY COOPERATION FOUNDATION, Seoul (KR)

(72) Inventors: Sukmook LEE, Seoul (KR); Yea Bin CHO, Gyeonggi-do (KR); Ji Woong KIM, Seoul (KR); Kyun HEO, Seoul (KR); Hyun Jung KIM, Seoul (KR)

(21) Appl. No.: 19/114,039

(22) PCT Filed: Sep. 25, 2023
(86) PCT No.: PCT/KR2023/014612

§ 371 Date: Mar. 21, 2025

(30) Foreign Application Priority Data
Sep. 23, 2022 (KR) 10-2022-0121201

(51) Int. Cl. A61K 47/68; A61P 35/04; C07K 16/18; C12N 15/85

(52) U.S. Cl. CPC A61K 47/68033; A61K 47/6843; A61P 35/04; C07K 16/18; C12N 15/85

(57) ABSTRACT

The present invention relates to: an antibody-drug conjugate comprising a drug and an antibody specifically binding to GRP94 or an antigen-binding fragment thereof; and a pharmaceutical composition for treating cancer, comprising same as an active ingredient. The antibody-drug conjugate of the present invention comprises an antibody specifically targeting GRP94 and having strong internalization activity, and, as a result of conjugation with a cytotoxic agent, effectively inhibits the growth of cetuximab-resistant CRC cell lines including HT29, HCT116, HCT8 and LoVo cells, and thus can be effectively used as a therapeutic agent for various carcinomas in which GRP94 is overexpressed.

### TECHNICAL FIELD

The present disclosure was made with the support of the Ministry of Science and ICT of the Republic of Korea under Project Identification Number 1711153505 and Project Number 2019M3E5D5065844. The research management agency for the project is the National Research Foundation of Korea, the research program is the “Bio & Medical Technology Development Program”, and the research project title is “Development of the next-generation therapeutic antibody for treating patients with cetuximab-resistant colorectal cancers.” The host institution is Kookmin University, and the research period was from Jan. 1, 2022, to Dec. 31, 2022.

This patent application claims priority to Korean Patent Application No. 10-2022-0121201, filed with the Korean Intellectual Property Office on Sep. 23, 2022, the disclosure of which is incorporated herein by reference.

The present disclosure relates to an antibody specifically binding to GRP94 or an antigen-binding fragment thereof, an antibody-drug conjugate comprising the same, and a pharmaceutical composition for cancer treatment comprising the same as an active ingredient.

### BACKGROUND ART

Colorectal cancer (CRC) is the third most common cancer worldwide and the fourth leading cause of cancer-related deaths. A variety of chemotherapeutic agents, including 5-fluorouracil (5-FU), irinotecan, and oxaliplatin, as well as combination regimens such as FOLFOX (leucovorin, 5-FU, and oxaliplatin) and FOLFIRI (leucovorin, 5-FU, and irinotecan), have been used as standard treatments for CRC. However, despite their clinical efficacy, these chemotherapeutic agents inhibit DNA synthesis and/or disrupt the structure of microtubules, thereby causing various side effects such as alopecia, diarrhea, thrombocytopenia, and paresthesia.

Therapeutic antibodies are one of the most effective targeted cancer treatments. Since the approval of the murine anti-CD3 monoclonal antibody (OKT3) by the U.S. FDA, remarkable advancements in recombinant DNA technology have enabled the production of various humanized and fully human antibodies. In particular, cetuximab, a recombinant mouse/human chimeric monoclonal antibody targeting the epidermal growth factor receptor (EGFR), has been developed and is widely used in clinical settings for CRC treatment. However, as a monotherapy, cetuximab is not sufficiently effective, and its use in combination with FOLFIRI or FOLFOX regimens is recommended. Furthermore, cetuximab is effective in only about 10-20% of CRC patients, while resistance to cetuximab is observed in the remaining patients. This has highlighted the need for the development of new therapeutic targets and treatment strategies.

Meanwhile, antibody-drug conjugates (ADCs) have emerged as a new class of cancer therapy that combines the potency of small-molecule therapeutics with the targeting ability of antibodies. By integrating these two components into a single novel molecular entity, highly cytotoxic small-molecule drugs can be selectively delivered to target cancer tissues, thereby enhancing therapeutic efficacy while reducing the potential systemic toxicity of the small-molecule drugs. In the production of ADCs, site-specific conjugation has been recognized as a strategy to overcome the drawbacks of first-generation ADCs, such as heterogeneity and the formation of unstable bioconjugates. The present inventors engineered an antibody specific to GRP94 or an antigen-binding fragment thereof to possess a selenocysteine residue at the C-terminus of each heavy chain and conjugated a drug with a stable linker to this engineered antibody. Through this approach, the inventors confirmed the potential for more effective treatment of cetuximab-resistant CRC.

Throughout this specification, numerous scientific papers and patent documents are cited and referenced. The disclosures of the cited papers and patent documents are incorporated herein by reference in their entirety to provide a clearer understanding of the level of ordinary skill in the art and the details of the present disclosure.

### DISCLOSURE OF INVENTION

### Technical Problem

The present inventors have developed an antibody specifically binding to GRP94 and conducted extensive research to establish a therapeutic strategy with greater precision and enhanced efficacy. As a result, the inventors engineered this antibody to include a selenocysteine residue at the C-terminus of each heavy chain and conjugated a drug with a stable linker to the engineered antibody. Through this approach, they confirmed the potential for more effective treatment of cetuximab-resistant CRC, ultimately resulting in the completion of the present disclosure.

Accordingly, an objective of the present disclosure is to provide an antibody specifically binding to GRP94 or an antigen-binding fragment thereof, and an antibody-drug conjugate (ADC) comprising a drug.

Another objective of the present disclosure is to provide a pharmaceutical composition comprising the aforementioned ADC as an active ingredient for cancer treatment, inhibition of cancer metastasis, or inhibition of angiogenesis.

Additional objectives and advantages of the present disclosure will become more apparent from the following detailed description, claims, and drawings.

### Solution to Problem

According to one aspect of the present disclosure, provided are:
(i) an antibody specifically binding to GRP94 or an antigen-binding fragment thereof, comprising HCDR1 having the amino acid sequence of SEQ ID NO: 1, HCDR2 having the amino acid sequence of SEQ ID NO: 2, HCDR3 having the amino acid sequence of SEQ ID NO: 3, LCDR1 having the amino acid sequence of SEQ ID NO: 4, LCDR2 having the amino acid sequence of SEQ ID NO: 5, and LCDR3 having the amino acid sequence of SEQ ID NO: 6; or (ii) an antibody specifically binding to GRP94 or an antigen-binding fragment thereof, comprising HCDR1 having the amino acid sequence of SEQ ID NO: 9, HCDR2 having the amino acid sequence of SEQ ID NO: 10, HCDR3 having the amino acid sequence of SEQ ID NO: 11, LCDR1 having the amino acid sequence of SEQ ID NO: 12, LCDR2 having the amino acid sequence of SEQ ID NO: 13, and LCDR3 having the amino acid sequence of SEQ ID NO: 14; and an antibody-drug conjugate (ADC) comprising the same and a drug.

In one embodiment of the present disclosure, the antibody or antigen-binding fragment of (i) comprises a heavy chain variable region having the amino acid sequence of SEQ ID NO: 7 and a light chain variable region having the amino acid sequence of SEQ ID NO: 8.

In another embodiment of the present disclosure, the antibody or antigen-binding fragment of (ii) comprises a heavy chain variable region having the amino acid sequence of SEQ ID NO: 15 and a light chain variable region having the amino acid sequence of SEQ ID NO: 16.

The antibody or antigen-binding fragment of (i) and (ii) according to an embodiment of the present disclosure are designated as “K101.1” and “K101.3” in this specification.

In this specification, the term “GRP94 (glucose-regulated protein 94)” refers to a chaperone protein encoded by the HSP90B1 gene, also known as GP96, ERp99, or endoplasmin. GRP94 is known to play a critical role in protein folding in secretory pathways, such as through Toll-like receptors and integrins, and is recognized as an essential immune chaperone that regulates both innate and adaptive immunity. GRP94 is also known as a therapeutic target in diseases such as glaucoma, multiple myeloma, and metastatic cancer.

In this specification, the term “antibody” refers to a specific antibody against GRP94, which includes not only the complete antibody form but also the antigen-binding fragments of the antibody molecule.

A complete antibody consists of two full-length light chains and two full-length heavy chains, where each light chain is linked to a heavy chain by a disulfide bond. The constant region of the heavy chain has gamma (γ), mu (μ), alpha (α), delta (δ), and epsilon (ε) types, with subclasses gamma1 (γ1), gamma2 (γ2), gamma3 (γ3), gamma4 (γ4), alpha1 (α1), and alpha2 (α2). The constant region of the light chain has kappa (κ) and lambda (λ) types (Cellular and Molecular Immunology, Wonsiewicz, M. J., Ed., Chapter 45, pp. 41-50, W. B. Saunders Co. Philadelphia, PA (1991); Nisonoff, A., Introduction to Molecular Immunology, 2nd Ed., Chapter 4, pp. 45-65, Sinauer Associates, Inc., Sunderland, MA (1984)).

In this specification, the term “antigen-binding fragment” refers to a fragment that retains antigen-binding functionality, including Fab, F(ab′), F(ab′)2, chemically linked F(ab′)2, and Fv. Among the antibody fragments, Fab has a structure consisting of the variable regions of the light and heavy chains, the constant region of the light chain, and the first constant region of the heavy chain (CH1), providing a single antigen-binding site. Fab′ differs from Fab in that it contains a hinge region at the C-terminal of the CH1 domain of the heavy chain, which includes one or more cysteine residues. F(ab′)2 antibodies are formed when the cysteine residues in the hinge region of Fab′ form disulfide bonds. Fv is the smallest antibody fragment, consisting only of the variable regions of the heavy and light chains. The recombinant technology for generating Fv fragments is disclosed in PCT international patent applications WO 88/10649, WO 88/106630, WO 88/07085, WO 88/07086, and WO 88/09344. Two-chain Fv (di-chain Fv) is a structure where the variable regions of the heavy and light chains are connected by non-covalent interactions, while single-chain Fv (scFv) is typically linked via a peptide linker, either covalently connecting the variable regions of the heavy and light chains or directly linked at the C-terminus, forming structures similar to dimers, like two-chain Fv. These antibody fragments can be obtained using protein hydrolyzing enzymes (for example, Fab can be obtained by digesting the whole antibody with papain, and F(ab′)2 fragments can be obtained with pepsin), or they can be produced through recombinant DNA technology.

In the present invention, the antibody is preferably in the form of scFv or a complete antibody. Additionally, the constant region of the heavy chain can be selected from any of the isotypes gamma (γ), mu (μ), alpha (α), delta (δ), or epsilon (ε). Preferably, the constant region is gamma1 (IgG1), gamma3 (IgG3), or gamma4 (IgG4), with the most preferred isotype being gamma1 (IgG1). The constant region of the light chain can be either kappa or lambda, with the kappa form being preferred. Therefore, the preferred antibody of the present invention is an scFv form or IgG1 form with a kappa light chain and a gamma1 heavy chain.

In this specification, the term “heavy chain” refers to both the full-length heavy chain, which includes the variable region domain VH with an amino acid sequence having sufficient variability to provide antigen specificity, and the three constant region domains CH1, CH2, and CH3, as well as its fragments. Additionally, in this specification, the term “light chain” refers to both the full-length light chain, which includes the variable region domain VL (Vk) with an amino acid sequence having sufficient variability to provide antigen specificity, and the constant region domain CL (Ck), as well as its fragments.

In this specification, the term “CDR (complementarity determining region)” refers to the amino acid sequences of the hypervariable regions of the heavy and light chains of immunoglobulins (Kabat et al., Sequences of Proteins of Immunological Interest, 4th Ed., U.S. Department of Health and Human Services, National Institutes of Health (1987)). The heavy chain (HCDR1, HCDR2, and HCDR3) and light chain (LCDR1, LCDR2, and LCDR3) each contain three CDRs. The CDRs provide the key contact residues for the antibody to bind to the antigen or epitope.

The antibody of the present invention includes, but is not limited to, monoclonal antibodies, multispecific antibodies (e.g., bispecific antibodies), human antibodies, humanized antibodies, chimeric antibodies, single-chain Fvs (scFv), single-chain antibodies, Fab fragments, F(ab′) fragments, disulfide-linked Fvs (sdFv), anti-idiotype (anti-Id) antibodies, and epitope-binding fragments of the above antibodies.

In this specification, the term “framework” or “FR” refers to the variable domain residues other than the hypervariable region (HVR) residues. The framework of a variable domain typically consists of four FR domains: FR1, FR2, FR3, and FR4. Therefore, the HVR and FR sequences generally appear in the following order in VH (or VL/Vk):
(a) FRH1 (Framework region 1 of Heavy chain)—HCDR1 (complementarity determining region 1 of Heavy chain)—FRH2-HCDR2-FRH3-HCDR3-FRH4; and
(b) FRL1 (Framework region 1 of Light chain)—LCDR1 (complementarity determining region 1 of Light chain)—FRL2-LCDR2-FRL3-LCDR3-FRL4.

The term “variable region” or “variable domain” refers to the domain of the antibody heavy chain or light chain that is involved in binding the antibody to the antigen. The variable domains of the native antibody heavy chain (VH) and light chain (VL) generally share similar structures, with each domain containing four conserved framework regions (FR) and three hypervariable regions (HVR) (Kindt et al., Kuby Immunology, 6th Edition, W.H. Freeman and Co., page 91, 2007). A single VH or VL domain can be sufficient to confer antigen-binding specificity. Furthermore, an antibody that binds to a specific antigen can be isolated by screening an antibody library of complementary VH or VL domains, which bind to the antigen and are separated using these VH or VL domains.

In this specification, the term “specifically binds” or similar terms refers to the ability of an antibody, its antigen-binding fragment, or other constructs like scFv, to form a relatively stable complex with the antigen under physiological conditions. Specific binding can be characterized by a dissociation constant (KD) of at least about 1×10⁻⁶ M, preferably 1×10⁻⁷ M, and more preferably 1×10⁻⁸ M or less (a smaller KD indicates a stronger binding). Methods for determining whether two molecules specifically bind are well-known in the industry, including equilibrium dialysis and surface plasmon resonance, among others. However, an isolated antibody that specifically binds human GRP94 may exhibit cross-reactivity with GRP94 molecules from other species or different antigens.

In this specification, the term “affinity” refers to the strength of the non-covalent interaction between a single binding site of a molecule (e.g., an antibody) and its binding partner (e.g., an antigen). Unless otherwise specified, as used in this specification, “binding affinity” refers to the intrinsic binding affinity that reflects a 1:1 interaction between the members of the binding pair (e.g., antibody and antigen). The affinity of molecule Y and its partner Y is generally represented by the dissociation constant (K_{D}). Affinity can be measured using conventional methods known in the industry, including those described in this specification.

In this specification, the term “human antibody” refers to an antibody generated by human or human cells, or an antibody that possesses an amino acid sequence corresponding to the amino acid sequence of an antibody derived from a non-human source using human antibody repertoires or other human antibody coding sequences. This definition of a human antibody excludes humanized antibodies, which contain non-human antigen-binding residues.

In this specification, the term “chimeric” antibody refers to an antibody in which part of the heavy chain and/or light chain is derived from a specific source or species, while the remainder of the heavy chain and/or light chain is derived from a different source or species.

In this specification, the term “humanized antibody” refers to a chimeric immunoglobulin, or its immunoglobulin chain or fragment (such as Fv, Fab, Fab′, F(ab′)2, or other antigen-binding subfragments of the antibody), which contains the minimal sequence derived from a non-human (e.g., mouse) antibody's non-human immunoglobulin. In most cases, the humanized antibody has the complementarity-determining region (CDR) residues of the recipient antibody replaced by the CDR residues of a non-human species (donor antibody), such as mouse, rat, or rabbit, which possess the intended specificity, affinity, and capabilities. In some cases, the framework region (FR) residues of the human immunoglobulin are replaced by corresponding non-human residues. Additionally, humanized antibodies may contain residues not found in the recipient antibody or the imported CDR or framework sequences. These modifications are made to further enhance and optimize the antibody's performance. Generally, the humanized antibody will include at least one, and typically two, substantially all of the variable domains, where all or substantially all of the CDR regions in these domains correspond to the CDR regions of the non-human immunoglobulin, and all or substantially all of the FR regions have the sequence of the human immunoglobulin's FR regions. The humanized antibody will also include at least a portion, or substantially all, of the Fc region sequence from the human immunoglobulin.

The anti-GRP94 antibody or its antigen-binding fragment of the present invention may, as one skilled in the art would recognize, include variants of the amino acid sequence within a range that allows for specific recognition of GRP94. For example, changes may be made to the amino acid sequence of the antibody to improve its binding affinity and/or other biological properties. These modifications may include, for instance, deletions, insertions, and/or substitutions of amino acid residues in the antibody's sequence.

The variant is said to have “substantial similarity,” meaning that when two peptide sequences are optimally aligned using programs such as GAP or BESTFIT with default gap penalties, they share at least about 90% sequence identity, and more preferably at least about 95%, 98%, or 99% sequence identity. Ideally, the non-identical residue positions are different due to conservative amino acid substitutions. A “conservative amino acid substitution” refers to the replacement of an amino acid residue by another amino acid residue with a similar chemical property (e.g., charge or hydrophobicity) in its side chain (R group). Generally, conservative amino acid substitutions do not significantly alter the functionality of the protein. When two or more amino acid sequences differ due to conservative substitutions, the percentage or degree of similarity may be adjusted upwards to account for the conservative nature of the substitutions.

These amino acid variations are made based on the relative similarity of the amino acid side chain substitutions, such as hydrophobicity, hydrophilicity, charge, size, etc. Through analysis of the size, shape, and type of amino acid side chain substitutions, it can be determined that arginine, lysine, and histidine all carry positively charged residues; alanine, glycine, and serine share similar sizes; and phenylalanine, tryptophan, and tyrosine have similar shapes. Based on these considerations, arginine, lysine, and histidine; alanine, glycine, and serine; and phenylalanine, tryptophan, and tyrosine can be considered biologically equivalent analogs.

In introducing mutations, the hydrophobicity index of amino acids can be considered. Each amino acid is assigned a hydrophobicity index based on its hydrophobicity and charge: Isoleucine (+4.5); Valine (+4.2); Leucine (+3.8); Phenylalanine (+2.8); Cysteine/Cystine (+2.5); Methionine (+1.9); Alanine (+1.8); Glycine (−0.4); Threonine (−0.7); Serine (−0.8); Tryptophan (−0.9); Tyrosine (−1.3); Proline (−1.6); Histidine (−3.2); Glutamate (−3.5); Glutamine (−3.5); Aspartate (−3.5); Asparagine (−3.5); Lysine (−3.9); and Arginine (−4.5).

In conferring the interactive biological function of a protein, the hydrophobicity index of amino acids is crucial. It is a well-known fact that substituting with amino acids having similar hydrophobicity indexes results in retaining similar biological activity. When introducing mutations based on the hydrophobicity index, it is preferable to substitute between amino acids with a hydrophobicity index difference of within ±2, more preferably within ±1, and even more preferably within ±0.5.

On the other hand, it is well known that substitutions between amino acids with similar hydrophilicity values result in proteins with equivalent biological activity. As disclosed in U.S. Pat. No. 4,554,101, the following hydrophilicity values are assigned to the respective amino acid residues: Arginine (+3.0); Lysine (+3.0); Aspartate (+3.0±1); Glutamate (+3.0±1); Serine (+0.3); Asparagine (+0.2); Glutamine (+0.2); Glycine (0); Threonine (−0.4); Proline (−0.5±1); Alanine (−0.5); Histidine (−0.5); Cysteine (−1.0); Methionine (−1.3); Valine (−1.5); Leucine (−1.8); Isoleucine (−1.8); Tyrosine (−2.3); Phenylalanine (−2.5); Tryptophan (−3.4).

When introducing mutations with reference to hydrophilicity values, it is preferable to substitute amino acids showing a difference in hydrophilicity value within +2, more preferably within ±1, and even more preferably within +0.5.

Amino acid exchanges in proteins that do not significantly alter the overall activity of the molecule are known in the art (H. Neurath, R. L. Hill, The Proteins, Academic Press, New York, 1979). The most commonly occurring exchanges are between amino acid residues such as Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly.

In one embodiment of the present invention, the antibody or antigen-binding fragment has a dissociation constant (K_{D}) value of 10⁻⁸ M or less.

In another embodiment of the present invention, the antibody or antigen-binding fragment has the efficacy to inhibit the proliferation of one or more tumor cell lines selected from the group consisting of HCT8, HT29, LoVo, HCT116, and Caco-2, both in vitro and in vivo.

In one embodiment of the present invention, the antibody or antigen-binding fragment of the present invention binds to a GRP94 target antigen expressed on or within the cell membrane of cancer cells, and after binding to the GRP94 target antigen, the antibody is internalized into the cell. This internalization property indicates that the antibody targeting GRP94 not only has potential as a therapeutic agent but also has the potential to effectively deliver drugs into cancer cells when applied as an antibody-drug conjugate.

As described above, the antibody-drug conjugate according to one embodiment of the present invention can be produced by conjugating the GRP94-specific antibody or its antigen-binding fragment of the present invention with a drug.

In one embodiment of the present invention, the antibody or its antigen-binding fragment is characterized by the conjugation of the drug to the constant region.

In a specific embodiment of the present invention, the constant region to which the drug is conjugated may be the Fc region of the antibody or antigen-binding fragment.

In one embodiment of the present invention, the antibody or its antigen-binding fragment may include selenocysteine in the constant region, such as the Fc region.

The selenocysteine may be included by substituting a cysteine in the constant region of the antibody or antigen-binding fragment, or may be additionally included in the Fc region, etc.

In this specification, “selenocysteine (Sec)” is a naturally occurring amino acid found in certain enzymes such as glutathione peroxidase. Selenocysteine has a structure similar to cysteine, except that the sulfur atom of cysteine is replaced by selenium. Therefore, proteins containing one or more selenocysteine residues are referred to as selenoproteins, as they contain selenium. Unlike other amino acids, selenocysteine is not directly encoded by the genetic code, but the UGA codon, which is normally recognized as a stop codon, can specify selenocysteine when a SECIS (selenocysteine insertion sequence) element is present on the mRNA.

The selenocysteine (Sec) can be strategically introduced to allow for site-specific conjugation of the payload in antibody-drug conjugates (ADCs). The selenocysteine residue offers a higher reactivity compared to the conventional cysteine residue used for site-specific ADC generation, enabling faster and single-step reactions under physiological conditions. The site-specific conjugation technology developed by introducing selenocysteine residues has been named “Selenomab.”

Therefore, in one embodiment of the present invention, the drug may be conjugated to the selenocysteine included in the antibody or antigen-binding fragment, but is not limited thereto.

In another embodiment of the present invention, the drug may be conjugated to the lysine residue of the antibody or its antigen-binding fragment, but is not limited thereto.

In the case where the antibody or antigen-binding fragment of the present invention is prepared as an antibody-drug conjugate, the drug (payload) may be conjugated to the GRP94 antibody or antigen-binding fragment of the present invention via a covalent bond through a chemical linker.

Therefore, the antibody-drug conjugate of the present invention comprises a linker to connect the antibody or its antigen-binding fragment to the drug.

In the present specification, the “linker” refers to any moiety that links, connects, or conjugates the antibody or its antigen-binding fragment to the drug described in the present application. Generally, a suitable linker for the antibody conjugate is one that is sufficiently stable to utilize the circulating half-life of the antibody, while also being capable of releasing the drug after the antigen-mediated internalization of the conjugate.

In one embodiment of the present invention, the linker may be a cleavable linker or a non-cleavable linker. A cleavable linker is one that is cleaved by intracellular metabolism, such as hydrolysis, reduction, or enzymatic reactions, and is then internalized. A non-cleavable linker is one that, after releasing the attached drug through the lysosomal degradation of the antibody, is internalized. Suitable linkers include, but are not limited to, acid-degradable linkers, enzymatically cleavable linkers, reduction-degradable linkers, self-immolative linkers, and non-cleavable linkers. Suitable linkers also include, but are not limited to, peptide linkers, glucuronide, succinimide-thioether, polyethylene glycol (PEG) units, hydrazones, maleimide-caproyl units, dipeptide units, valine-citrulline units, and para-amino-benzyl (PAB) units, or those containing them.

In one embodiment of the present invention, the linker may be (6-maleimidocaproyl) hydrazone, (4-(4′-acetylphenoxy) butanoic acid) hydrazone, SMCC (N-succinimidyl-4-(maleimidomethyl)cyclohexane-1-carboxylate), Fmoc-Val-Cit-PAB-OH, Fmoc-Val-Cit-PAB-PNP, Mc-Val-Cit-PABC-PNP, Val-Cit-PAB, or MC-Val-Cit-PAB-PNP, but is not limited to these.

In one embodiment of the present invention, the drug may be a cytotoxic agent. The cytotoxic agent refers to a substance that inhibits or prevents the expression or function of cells and/or causes the destruction of cells. This term encompasses toxins such as radioactive isotopes, chemotherapeutic agents, and enzymatically active toxins or low molecular weight toxins derived from bacteria, fungi, plants, or animals, as well as their fragments and/or variants.

Examples of cytotoxic agents include auristatin (e.g., auristatin E, auristatin F, MMAE, and MMA), auromycin, maytansinoids, ricin, ricin A-chain, combretastatin, duocarmycin, dolastatin, doxorubicin, daunorubicin, paclitaxel, cisplatin, CC1065, ethidium bromide, mitomycin, etoposide, teniposide, vincristine, vinblastine, colchicine, dihydroxyanthraquinone, actinomycin, diphtheria toxin, Pseudomonas exotoxin (PE) A, PE40, abrin, abrin A-chain, modecine A-chain, α-sarcin, gelonin, mitogelin, restrictocin, phenomycin, enomycin, curicin, crotonin, calicheamicin, Saponaria officinalis inhibitor, glucocorticoids, and other chemotherapeutic agents, as well as radioactive isotopes such as At²¹¹, I¹³¹, I¹²⁵, Y⁹⁰, Re¹⁸⁶, Re¹⁸⁸, Sm¹⁵³, Bi²¹², or Bi²¹³, P³², and Lu¹⁷⁷, including radioactive isotopes of Lu, but not limited to these.

In a specific embodiment of the present invention, the cytotoxic agent is a maytansinoid. More specifically, the maytansinoid is DM1 or DM4, a tomeimysin derivative, or a dolastatin derivative. Most specifically, the maytansinoid is DM1.

In a specific embodiment of the present invention, the cytotoxic agent is auristatin. More specifically, the auristatin is MMAE, MMAF, or a derivative thereof. Most specifically, the auristatin is MMAE.

In an embodiment of the present invention, the antibody-drug conjugate of the present invention includes 1 to 20 units of the drug per antibody or antigen-binding fragment thereof. More specifically, the antibody-drug conjugate includes 1 to 20, 1 to 16, 1 to 12, 1 to 10, 1 to 9, 1 to 8, 1 to 7, 1 to 6, 1 to 5, 1 to 4, 1 to 3, or 1 to 2 units of the drug per antibody or antigen-binding fragment thereof.

The unit per antibody or antigen-binding fragment thereof can be an integer or a non-integer. The unit per antibody or antigen-binding fragment thereof is measured as the average ratio of drug to antibody. For example, if on average two DM1 molecules are conjugated per antibody or antigen-binding fragment, the unit per antibody or antigen-binding fragment would be 2.

According to another aspect of the present invention, the invention provides a pharmaceutical composition for cancer treatment, cancer metastasis inhibition, or angiogenesis inhibition, comprising the antibody-drug conjugate, which includes the antibody or antigen-binding fragment specifically binding to GRP94 as described above and a drug as an active ingredient.

In this specification, the term “angiogenesis” refers to the process by which new capillaries are formed from existing microvessels. When angiogenesis occurs normally, it takes place during embryonic development, tissue regeneration, and wound healing, as well as during cyclical changes in the female reproductive system when the corpus luteum develops, and these processes are strictly regulated even in these cases (Folkman J et al., Int. Rev. Exp. Pathol., 16, pp207-248, 1976). In adults, endothelial cells grow very slowly and, compared to other types of cells, they relatively infrequently divide. The process of angiogenesis generally involves the degradation of the vascular basement membrane by proteases, migration, proliferation, and differentiation of endothelial cells, leading to the formation of lumens and reconstruction of the vessels, which results in the creation of new capillaries. However, there are diseases caused when angiogenesis is not autonomously regulated and grows pathologically.

Diseases related to angiogenesis in pathological conditions include hemangiomas, hemangiofibromas, vascular malformations, and cardiovascular diseases such as atherosclerosis, vascular adhesions, and edematous sclerosis. Ocular diseases related to angiogenesis include corneal graft-induced angiogenesis, neovascular glaucoma, diabetic retinopathy, corneal diseases caused by neovascularization, macular degeneration, pterygium, retinal degeneration, age-related macular degeneration, posterior segment fibrosis, and granulomatous conjunctivitis.

Chronic inflammatory diseases such as arthritis, psoriasis, telangiectasia, pyogenic granulomas, seborrheic dermatitis, acne, dermatological diseases, as well as Alzheimer's disease and obesity, are also related to angiogenesis, and the growth and metastasis of cancer depend on angiogenesis (D'Amato R J et al., Ophthalmology, 102 (9), pp1261-1262, 1995; Arbiser J L, J. Am. Acad. Dermatol., 34 (3), pp486-497, 1996; O'Brien K D et al., Circulation, 93 (4), pp672-682, 1996; Hanahan D et al., Cell, 86, pp353-364, 1996).

In particular, in cancer, angiogenesis plays a crucial role in the growth and metastasis of cancer cells. Tumors receive the nutrients and oxygen necessary for growth and proliferation through newly formed blood vessels, and the newly formed vessels that penetrate the tumor also provide a route for metastatic cancer cells to enter the bloodstream, facilitating their spread (Folkman and Tyler, Cancer Invasion and Metastasis, Biologic Mechanisms and Therapy (S.B. Day ed.), Raven Press, New York, pp94-103, 1977; Polverini P J, Crit. Rev. Oral. Biol. Med., 6 (3), pp230-247, 1995). The primary cause of death in cancer patients is metastasis, and the reason why current chemotherapy and immunotherapies do not significantly improve the survival rate of cancer patients is due to the metastasis of cancer.

Arthritis, a representative inflammatory disease, is caused by autoimmune abnormalities, but as the disease progresses, chronic synovial inflammation induces angiogenesis, leading to cartilage destruction. In other words, with the help of cytokines that induce inflammation, synoviocytes and endothelial cells proliferate in the synovial cavity, and as angiogenesis progresses, the pannus, a connective tissue layer that forms in the cartilage, destroys the cushioning cartilage (Koch A E et al., Arthritis. Rheum., 29, pp471-479, 1986; Stupack D G et al., Braz J. Med. Biol. Res., 32 (5), pp578-581, 1999; Koch A E, Arthritis. Rheum., 41 (6), pp951-962, 1998).

Each year, millions of people around the world become blind due to various ocular diseases, many of which are caused by angiogenesis (Jeffrey M I et al., J. Clin. Invest., 103, pp1231-1236, 1999). Representative examples of such diseases include age-related macular degeneration, diabetic retinopathy, retinopathy of prematurity, neovascular glaucoma, and corneal diseases caused by neovascularization (Adamis A P et al., Angiogenesis, 3, pp9-14, 1999). Among these, diabetic retinopathy is a complication of diabetes where the capillaries in the retina are invaded by the vitreous, eventually leading to blindness.

Psoriasis, a chronic proliferative disease of the skin characterized by red patches and scales, is another condition in which angiogenesis plays a crucial role. It is an incurable disease that causes pain and deformities. In normal individuals, keratinocytes proliferate once a month, but in psoriasis patients, this process occurs at least once a week. To support this rapid proliferation, a substantial blood supply is required, which leads to active angiogenesis (Folkman J, J. Invest. Dermatol., 59, pp40-48, 1972).

In the present invention, the term “cancer metastasis” refers to the process by which tumor cells move from one organ or region to another that is distantly located. The metastasis of cancer occurs through a series of stages: the invasion phase, where the in situ tumor infiltrates the basement membrane; the intravasation phase, where cancer cells that have crossed the basement membrane enter the circulatory system through blood vessels or lymphatic vessels; and the metastatic colonization phase, where cancer cells that have survived after circulating through the bloodstream are captured in other organs, possibly going through a dormant single-cell or latent micro-metastasis period, ultimately leading to the formation of metastatic colonies along with the development of new blood vessels.

In one embodiment of the present invention, the cancer includes one or more cells that express GRP94 at an increased level compared to non-cancerous cells.

In a more specific embodiment of the present invention, the cancer includes solid tumors or hematological malignancies.

As used in this specification, the term “solid tumor” refers to a mass of cancer that arises from abnormal cell growth in various solid organs (such as the bladder, breast, colon, kidney, lung, liver, brain, esophagus, gallbladder, ovary, pancreas, stomach, cervix, thyroid, prostate, and skin), in contrast to hematological cancers.

In one embodiment of the present invention, the solid tumor includes one or more cancers selected from the group consisting of gastric cancer, rectal cancer, colon cancer, rectosigmoid cancer, inflammation-related colonic cancer, liver cancer, lung cancer (non-small cell lung cancer, adenocarcinoma of the lung), ovarian cancer, melanoma, pancreatic cancer, uterine cancer, testicular cancer, and breast cancer, but is not limited thereto (Wu et al., Adv Cancer Res. 2016; 129:165-90.; Ansa-Addo et al., Curr Top Med Chem. 2016; 16 (25): 2765-2778.).

In this specification, the term “hematologic cancer” refers to cancer that occurs in the components of blood, meaning malignant tumors that arise in blood, hematopoietic organs, lymph nodes, and lymphatic tissues.

In one specific embodiment of the present invention, the hematologic cancer includes one or more cancers selected from the group consisting of acute myeloid leukemia, acute lymphoblastic leukemia, chronic myeloid leukemia, chronic lymphocytic leukemia, acute monoblastic leukemia, multiple myeloma, Hodgkin lymphoma, and non-Hodgkin lymphoma, but is not limited thereto.

The pharmaceutically acceptable carrier included in the pharmaceutical composition of the present invention is one commonly used in formulation, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil, but is not limited thereto. The pharmaceutical composition of the present invention may additionally include lubricants, wetting agents, sweeteners, flavoring agents, emulsifiers, suspending agents, preservatives, and the like, in addition to the above components. Suitable pharmaceutically acceptable carriers and formulations are described in detail in Remington's Pharmaceutical Sciences (19th ed., 1995).

The pharmaceutical composition of the present invention can be administered orally or non-orally. For example, it can be administered through intravenous injection, subcutaneous injection, intramuscular injection, intraperitoneal injection, topical administration, intranasal administration, pulmonary administration, or rectal administration.

In one embodiment of the present invention, the administration is through intravenous administration, intravitreal administration, intrathecal administration, parenteral administration, subcutaneous administration, transdermal administration, or infusion.

The appropriate dosage of the pharmaceutical composition of the present invention may vary depending on factors such as formulation method, administration route, patient's age, weight, gender, medical condition, diet, administration time, administration pathway, excretion rate, and responsiveness. A skilled physician can easily determine and prescribe the effective dosage for the desired treatment or prevention. In a preferred embodiment of the present invention, the daily dosage of the pharmaceutical composition is 0.0001 to 100 mg/kg. The term “pharmaceutically effective amount” as used in this specification refers to an amount sufficient to treat or inhibit the aforementioned cancer, cancer metastasis, or angiogenesis.

The pharmaceutical composition of the present invention can be formulated into unit dose forms or filled into multi-dose containers using pharmaceutically acceptable carriers and/or excipients, in accordance with methods that can be easily carried out by those skilled in the art of the technology to which the present invention pertains. The formulation may be in the form of a solution, suspension, or emulsion in an oil or aqueous medium, or it may be in the form of a paste, powder, suppository, granules, tablets, or capsules. Additionally, the formulation may further include dispersing agents or stabilizers.

Since the pharmaceutical composition of the present invention utilizes the antibody-drug conjugate as the active ingredient, the common details between the two are omitted in order to avoid excessive complexity in the specification caused by repetitive descriptions.

### Advantageous Effects of Invention

The features and advantages of the present invention can be summarized as follows:
(a) The present invention provides an antibody-drug conjugate comprising an antibody or its antigen-binding fragment that specifically binds to GRP94 and a drug, as well as a pharmaceutical composition for cancer treatment containing the same as an active ingredient.
(b) The antibody-drug conjugate of the present invention includes an antibody that specifically targets GRP94 and has strong internalization activity. As a result of conjugation with a cytotoxic agent, it effectively inhibits the growth of cetuximab-resistant colon-rectal cancer cell lines, such as HT29, HCT116, HCT8, and LoVo, and can be usefully employed as a therapeutic agent for treating various cancers that overexpress GRP94.

### BRIEF DESCRIPTION OF THE DRAWINGS

FIG. 1 illustrates the internalization ability of the GRP94-specific antibody K101.1 of the present invention, comparing its internalization characteristics with those of the anti-HER2 internalization antibody trastuzumab.

FIGS. 2a and 2b show the recombinant vector encoding K101.1-Sec, which was constructed to introduce selenocysteine (Sec) into the GRP94-specific antibody K101.1.

FIG. 3 displays the expression and purification of the proteins K101.1-Sec (SelT) and K101.1-Sec (TXNRD1).

FIG. 4 compares the expression yields of the proteins K101.1-Sec (SelT) and K101.1-Sec (TXNRD1).

FIG. 5 is a schematic diagram illustrating the method of conjugating K101.1-Sec (SelT) with commercially available SMCC-DM1 under mildly acidic conditions to generate the selenomab ADC.

FIG. 6 shows the yield of K101.1-Sec-DM1.

FIG. 7 confirms the expression of selenocysteine by detecting the HA tag expression of K101.1-Sec-DM1 using ELISA.

FIG. 8 compares the binding ability of the selenomab ADC to the target antigen by evaluating the binding of the monoclonal antibody K101.1 and its antibody-drug conjugate K101.1-Sec-DM1 to immobilized recombinant human/mouse GRP94 (rhGRP94, rrGRP94) using ELISA.

FIG. 9 shows the effect of the antibody-drug conjugate K101.1-Sec-DM1 and the monoclonal antibody K101.1 on the growth of CRC cells.

FIG. 10 illustrates the internalization effect of the antibodies (K101.1 and K101.3) on CRC cells, comparing the degree of antibody internalization with the positive control trastuzumab.

FIG. 11 evaluates the antitumor activity of the antibody-drug conjugates (K101.1-DM1 and K101.3-DM1) on CRC cells by culturing HCT116, HT29, HCT8, and LoVo cells with the antibody-drug conjugates and assessing the cell viability of CRC cells.

FIGS. 12 and 13 show the efficacy of K101.1, K101.3, K101.1-DM1, and K101.3-DM1 in evaluating antitumor activity on CRC cell spheroid formation. These figures demonstrate the effectiveness of the antibody-drug conjugates in inhibiting the growth of CRC cell spheroids.

### MODE FOR CARRYING OUT THE INVENTION

The following examples are provided to further explain the present invention in more detail. These examples are solely intended to illustrate the present invention more specifically, and it will be obvious to those skilled in the art that the scope of the present invention is not limited by these examples according to the gist of the invention.

### EXAMPLES

Throughout the specification, “%” used to denote the concentration of specific substances, unless otherwise stated, refers to weight/weight (%) for solid/solid, weight/volume (%) for solid/liquid, and volume/volume (%) for liquid/liquid.

### Experimental Methods and Materials (Example 1)

### 1-1. Cell Culture

HT29, LoVo, and HCT116 cell lines, which are human metastatic CRC cell lines (Korean Cell Line Bank, Seoul, Korea), were maintained in Roswell Park Memorial Institute 1640 (RPMI 1640) medium (Gibco, Invitrogen, Grand Island, NY, USA). The medium was supplemented with 10% (v/v) fetal bovine serum (FBS, Gibco) and 1% (v/v) penicillin/streptomycin (Gibco). CRC cell lines were cultured at 37° C. in a humidified incubator with 5% CO₂. Expi293F cells (Gibco) were cultured at 37° C. and 8% CO₂ in Expi293 expression medium (Gibco) in a shaking incubator.

### 1-2. Measurement of Antibody Internalization

Antibody internalization in HCT116 cells was measured using the FabFluor-pH Red Antibody Labeling Reagent (Sartorius, Gottingen, Germany) according to the manufacturer's instructions. Specifically, the red fluorescence intensity of the FabFluor reagent is known to be very low under neutral or basic pH conditions (extracellular or on the cell surface), while it significantly increases in acidic conditions in the lumen of endosomes and lysosomes resulting from internalized antibody labeled by the reagent. HCT116 cells were seeded at a density of 10,000 cells/well in a 96-well culture plate and allowed to adhere overnight. The control IgG, K101.1, or trastuzumab (MedChemExpress, Princeton, NJ, USA) were individually labeled with the human FabFluor-pH Red antibody labeling reagent at a 1:3 molar ratio in PBS and incubated for 15 minutes. The labeled antibodies were added to the cell culture medium at a final concentration of 2 μg/ml. The plate was transferred to the Incucyte SX1 live cell analysis system (Sartorius) and images were captured with a 10× objective lens for 12 hours. The fluorescence intensity was individually measured for each image.

### 1-3. Generation of K101.1 Selenomab

K101.1 selenomab (K101.1-Sec) was designed to incorporate a selenocysteine residue at the C-terminus of each heavy chain. Specifically, the Fc region of the selenomab was constructed to include the hinge-CH1-CH2-CH3 sequence of human IgG1, followed by the TGA codon, an HA tag sequence, the TAA codon, and a selenocysteine insertion sequence (Sec incorporation sequence, SECIS) element. These elements are derived from the GGGA-type Toxoplasma gondii selenoprotein T (SelT) or the human selenoprotein thioredoxin reductase 1 (TXNRD1), resulting in the generation of K101.1-Sec (SelT) or K101.1-Sec (TXNRD1).

After transfecting Expi293F cells with each recombinant vector encoding K101.1 selenomab, the Expi293F cells were cultured in an Erlenmeyer flask (Corning, Steuben County, NY, USA) containing Expi293 expression medium (Gibco) supplemented with 1 μM sodium selenite (Na2SeO3) (Sigma-Aldrich, St. Louis, MO, USA). The cells were cultured under humidified conditions at 37° C., 8% CO₂, and shaken at 130 rpm.

The overproduced proteins were then purified from the culture medium using affinity column chromatography with Protein A Sepharose (Repligen, Waltham, MA, USA). Subsequently, 5 μg of K101.1-Sec (SelT), K101.1-Sec (TXNRD1), or K101.1 were separated under reducing conditions using sodium dodecyl sulfate-polyacrylamide gel electrophoresis (SDS-PAGE) with a 12% polyacrylamide gel. After electrophoresis, the bands of the gel were visualized by Coomassie Brilliant Blue staining.

### 1-4. Drug Conjugation to K101.1 Selenomab

For selective drug conjugation at the Sec residue, K101.1-Sec (SelT) was diluted in 15 mL of 100 mM sodium acetate (pH 5.2) and concentrated to a final concentration of 3.3 UM (0.5 mg/mL) using a 30 kDa cutoff centrifugation filter device. The protein was incubated at room temperature with 0.1 mM dithiothreitol (DTT) for 20 minutes, followed by incubation with 33 UM of SMCC-DM1 (Chem scene, Monmouth Junction, NJ, USA) for 1 hour at room temperature in the dark to reduce the disulfide bonds. DTT and unreacted compounds were removed using a PD-10 desalting column (GE Healthcare Life Sciences, Piscataway, NJ, USA). The resulting K101.1-Sec-DM1 conjugate was stored at 4° C. for further studies.

### 1-5. ELISA (Enzyme-Linked Immunosorbent Assay)

A 96-well microplate was coated with 0.1 μg of rhGRP94 and incubated at 4° C. for 16 hours. After blocking the plate with 3% (w/v) BSA in PBS at 37° C. for 2 hours, the plate was incubated with 20 g/mL of K101.1-Sec (SelT) at 37° C. for 2 hours. After washing the plate three times with PBS-T, it was incubated with HRP-conjugated anti-human Fc secondary antibody (1:5,000; Invitrogen) or HRP-conjugated anti-HA antibody (1:3,000; Bethyl Laboratories, Montgomery, TX, USA) at 37° C. for 1 hour. The color reaction was initiated with 100 μL of TMB substrate solution and terminated with 2N H₂SO₄ solution. Optical density was measured at 450 nm using a microplate reader.

A 96-well microplate was coated with 0.1 μg of rhGRP94 and incubated at 4° C. for 16 hours. After blocking with 3% (w/v) BSA in PBS at 37° C. for 2 hours, the plate was incubated with 20 μg/mL of K101.1-Sec (SelT) at 37° C. for 2 hours. After washing three times with PBS-T, the plate was incubated with HRP-conjugated anti-human Fc secondary antibody (1:5,000; Invitrogen) at 37° C. for 1 hour. The color reaction was initiated with 100 μL of TMB substrate solution and terminated with 2N H₂SO₄ solution. Optical density was measured at 450 nm using a microplate reader.

### 1-6. Cell Proliferation Assay

To investigate the effect of K101.1-Sec-DM1 on CRC cell growth in vitro, 1×10⁴ HT29, LoVo, or 5×10³ HCT116 CRC cells were seeded in the wells of a 96-well plate and incubated at 37° C. for 16 hours. The cells were then treated with either 133 nM of K101.1 or 33 nM and 133 nM of K101.1-Sec-DM1, with or without the presence of the drug. Cell growth was measured for 62 hours using the Incucyte SX1 live cell analysis instrument (Sartorius).

### Experimental Results (Example 1)

### Example 1-1: K101.1 Internalization Induces the Downregulation of Cell Surface GRP94 on HCT116 CRC Cells

The inventors conjugated the control IgG, trastuzumab, or K101.1 with the FabFluor reagent, and then treated HCT116 cells with each conjugate to monitor the internalization over time and quantitatively detect it. To investigate the internalization ability of K101.1 in HCT116 cells, trastuzumab, a well-known anti-HER2 internalizing antibody, was used as a positive control.

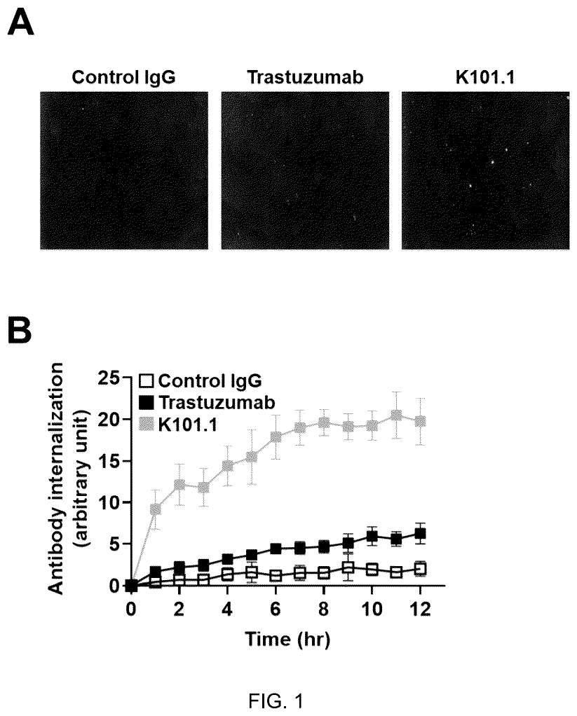

The results are shown in FIG. 1.

As shown in FIG. 1, the inventors found that K101.1 promoted internalization more efficiently than trastuzumab, whereas the control IgG did not induce internalization. This result indicates that K101.1 specifically induces internalization of GRP94 in the CRC cell line HCT116.

### Example 1-2: Generation of K101.1-Sec-DM1

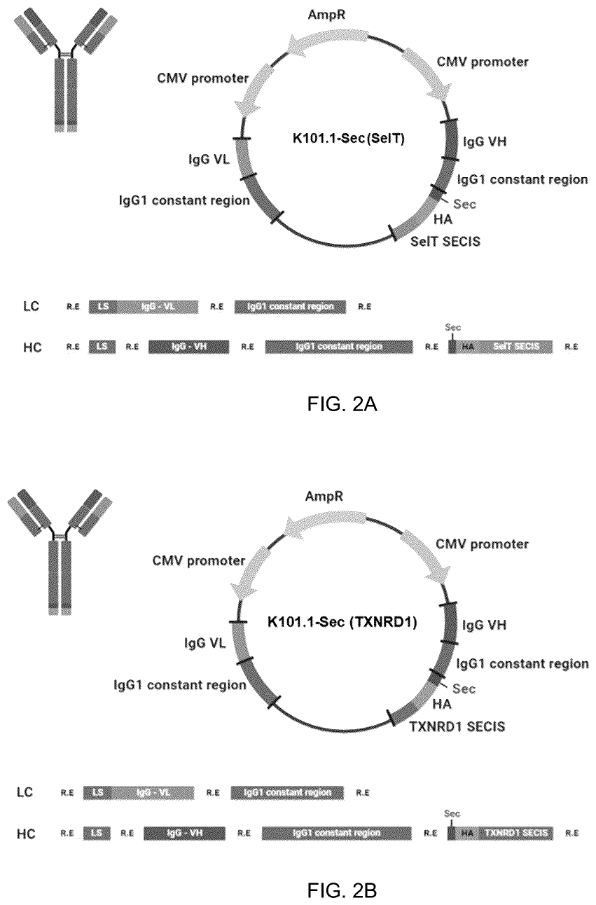

The inventors constructed each mammalian expression vector encoding the SECIS (Sec incorporation sequence) element from SelT or TXNRD1, along with a TGA codon, HA coding sequence, TAA stop codon, and C-terminal region of human IgG1 Fc, to express K101.1-Sec (FIGS. 2a and 2b).

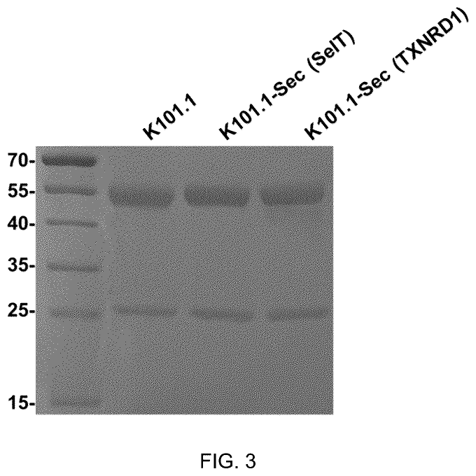

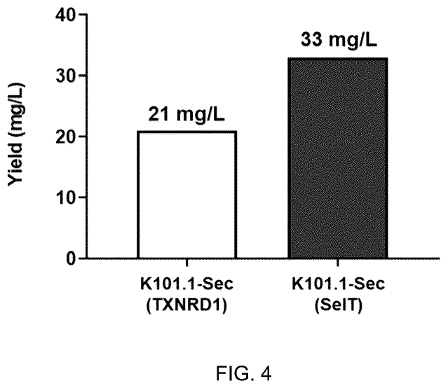

The recombinant vector encoding K101.1-Sec was transiently transfected into Expi293F cells maintained in Expi293 expression medium supplemented with 1 μM sodium selenite. Protein expression was induced in the cells, and K101.1-Sec was purified using protein A affinity chromatography. The results are shown in FIGS. 3 and 4.

As shown in FIG. 4, the yield of K101.1-Sec (SelT) reached 33 mg/L, while the yield of K101.1-Sec (TXNRD1) was 21 mg/L. Therefore, for subsequent experiments, K101.1-Sec (SelT) was used as the selenomab antibody drug conjugate (ADC) with the higher yield.

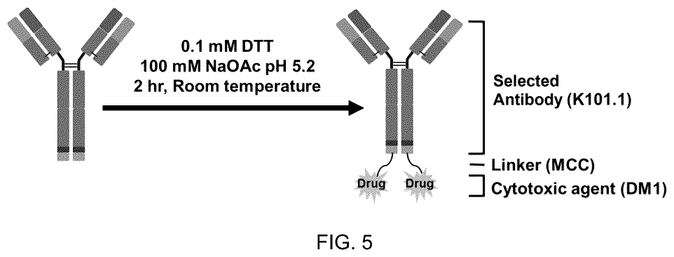

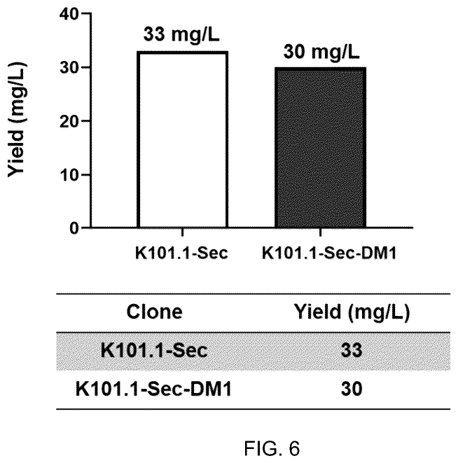

To generate the selenomab ADC, commercially available SMCC-DM1 was conjugated to K101.1-Sec (SelT) under weakly acidic conditions (FIG. 5). After conjugation, the inventors confirmed that the final yield of K101.1-Sec-DM1 was 30 mg/L (FIG. 6).

### Example 1-3: Confirmation of Selenocysteine Expression

The inventors confirmed the expression of selenocysteine in K101.1-Sec-DM1 by comparing it with the parent antibody, K101.1, using an HA tag.

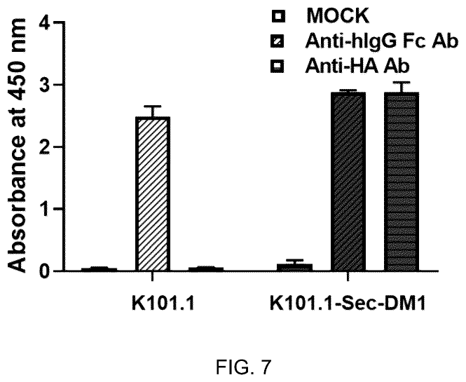

The results are shown in FIG. 7.

As depicted in FIG. 7, it was observed that while the parent antibody K101.1 did not express the HA tag, K101.1-Sec-DM1 did express the HA tag, indicating that selenocysteine was successfully expressed.

### Example 1-4: Binding Ability of K101.1-Sec-DM1 to GRP94 Antigen

The inventors evaluated the binding ability of the selenomab ADC to the target antigen using ELISA, comparing the binding of the parent antibody K101.1 and its antibody-drug conjugate K101.1-Sec-DM1 to immobilized rhGRP94 and rmGRP94.

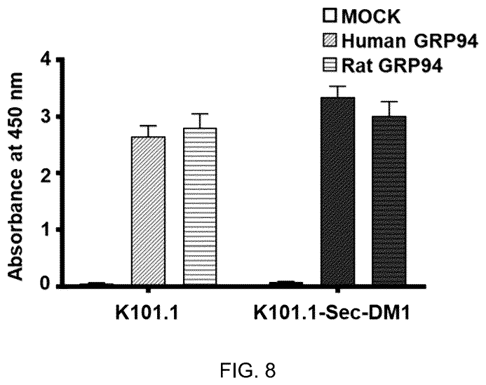

The results are shown in FIG. 8.

As depicted in FIG. 8, no significant difference in antigen binding was observed between the parent antibody K101.1 and the antibody-drug conjugate K101.1-Sec-DM1.

### Example 1-5: In Vitro Antitumor Effect of K101.1-Sec-DM1

The inventors evaluated the effect of K101.1-Sec-DM1 or K101.1 on CRC cell growth by culturing cells in the presence or absence of K101.1-Sec-DM1 or K101.1. CRC cell growth was monitored through real-time live cell imaging.

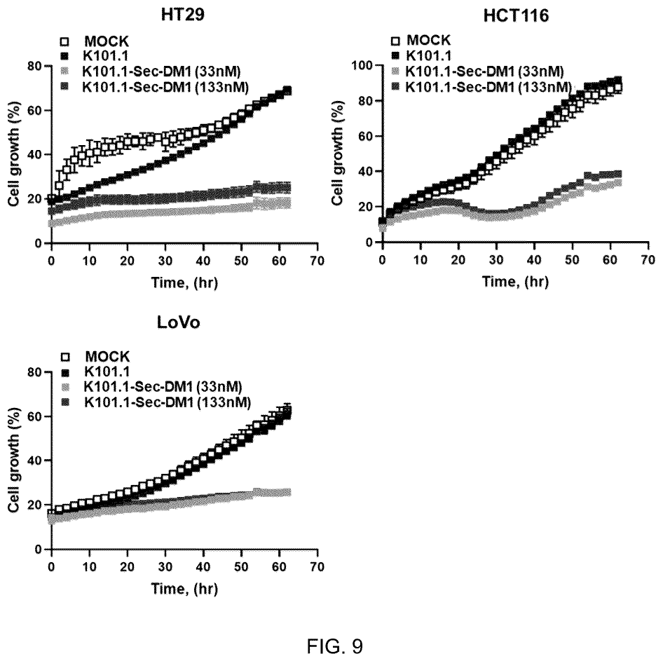

The results are shown in FIG. 9.

As depicted in FIG. 9, the inventors found that K101.1-Sec-DM1 significantly and more strongly inhibited the growth of HT29, LoVo, and HCT116 cell lines compared to K101.1, which showed little to no effect.

### Experimental Methods and Materials (Example 2)

### 2-1. Cell Culture

Human metastatic CRC cell lines (HCT8, HT29, LoVo, and HCT116) obtained from the Korean Cell Line Bank (Seoul, South Korea) were maintained in Roswell Park Memorial Institute 1640 medium (Gibco, Invitrogen, Grand Island, NY, USA) supplemented with 10% (v/v) fetal bovine serum (FBS, Gibco) and 1% (v/v) penicillin/streptomycin (Gibco). The CRC cell lines were cultured at 37° C. in a humidified incubator with 5% CO₂. Expi293F cells (Gibco) were cultured in Expi293 expression medium (Gibco) in a humidified shaking incubator at 37° C. with 8% CO₂.

### 2-2. Measurement of Antibody Internalization

The internalization of antibodies in HCT116 cells was measured using the FabFluor-pH Red antibody labeling reagent (Sartorius, Göttingen, Germany) according to the manufacturer's instructions. The FabFluor reagent exhibits very low fluorescence intensity at neutral or basic pH (outside the cell or on the cell surface). However, after internalization of the reagent-labeled antibody, the fluorescence intensity increases under the acidic conditions inside endosomes and lysosomes. HCT116 cells were seeded at a density of 10,000 cells per well in a 96-well culture plate and allowed to attach overnight. Antibodies K101.1, K101.3, or trastuzumab (MedChemExpress, Monmouth Junction, NJ, USA) were individually labeled using the FabFluor-pH Red antibody labeling reagent in a 1:3 molar ratio with phosphate-buffered saline (PBS) and incubated for 15 minutes. The labeled antibodies were added to the cell medium at a final concentration of 2 μg/mL. The plate was transferred to an Incucyte SX1 live cell analysis system (Sartorius), and images were captured for 10 hours using a 10× objective lens. Fluorescence intensity was then measured.

### 2-3. Antibody-Drug Conjugation

To generate the immunoglobulin G (IgG) form of the antibodies, the variable heavy chain (scFv) and light chain genes of the selected scFv clones were individually subcloned into the bicistronic mammalian expression vector pcDNA3.1 (Invitrogen). The two IgG antibodies (K101.1 and K101.3) were overproduced and purified using protein A Sepharose (Repligen, Waltham, MA, USA) affinity column chromatography. Next, to conjugate the drug to the lysine residues, K101.1 or K101.3 was diluted in conjugation buffer (100 mM sodium phosphate and 150 mM NaCl, pH 7.2) and concentrated to a final concentration of 13.3 μM (2 mg/mL) using a 30 kDa cutoff centrifugal filter device. The protein was incubated with 133 μM SMCC-DM1 (MedChemExpress) at 32° C. for 6 hours. Unreacted compounds were removed using a PD-10 desalting column (GE Healthcare Life Sciences, Piscataway, NJ, USA). The resulting K101.1-DM1 and K101.3-DM1 were stored at 4° C. until further use.

### 2-4. Cell Proliferation Assay

To evaluate the in vitro effects of K101.1-DM1 and K101.3-DM1 on CRC cell growth, HT29, LoVo, and HCT8 cells (seeded at a density of 1×10⁴ cells per well) and HCT116 cells (5×10³ cells per well) were plated in 96-well plates and treated with serial dilutions of K101.1-DM1 or K101.3-DM1. Cell viability was assessed using the Cell Counting Kit-8 (Sigma Aldrich, St. Louis, MO, USA) according to the manufacturer's instructions. The final absorbance was measured at 450 nm using a microplate reader (Synergy H1, BioTek).

### 2-5. Three-Dimensional Tumor Spheroid Assay

HCT116, HCT8, HT29, or LoVo cells were seeded at a density of 1,000 cells per well in a 96-well ultralow attachment round-bottom microplate (Thermo Fisher Scientific) using the previously described cell culture medium. After three days, when the spheroid size reached 200-250 μm, the spheroids were treated with 50 nM or 150 nM K101.1-DM1 or K101.3-DM1. Spheroid size was monitored for 10 days and quantified using ImageJ software (National Institutes of Health).

### Experimental Results (Example 2)

### Example 2-1. Analysis of the Internalization Effect of GRP94 Antibodies in HCT116 CRC Cells

The inventors investigated the internalization effect of K101.1 and K101.3 in HCT116 cells compared to trastuzumab. Using FabFluor reagent, trastuzumab, K101.1, and K101.3 were conjugated and subsequently applied to HCT116 cells to monitor and quantitatively detect antibody internalization over time. Trastuzumab, a well-established anti-human epidermal growth factor receptor 2 (HER2) antibody with strong internalization activity, was used as a positive control.

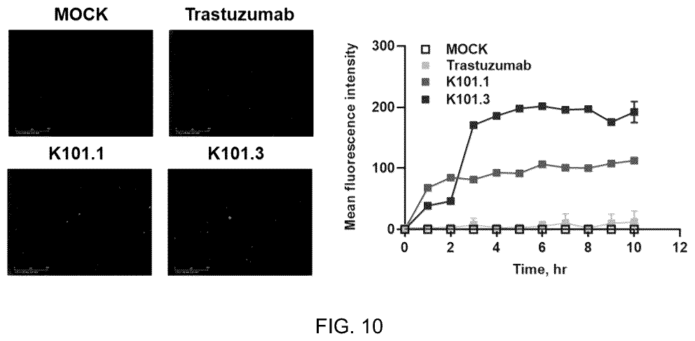

The results are shown in FIG. 10.

As depicted in FIGS. 10, K101.1 and K101.3 exhibited stronger internalization than trastuzumab. These findings suggest that K101.1 and K101.3 are highly specific and potent internalizing antibodies that could be utilized for the development of novel ADCs for CRC treatment.

### Example 2-2. Evaluation of the Antitumor Activity of GRP94 Antibody-Drug Conjugates in CRC Cells

The inventors assessed the in vitro effects of GRP94 ADCs (K101.1-DM1 and K101.3-DM1) on CRC cell growth by culturing HCT116, HT29, HCT8, and LoVo cells in the presence of these GRP94 ADCs. The viability of CRC cells was determined using a CCK-8 assay.

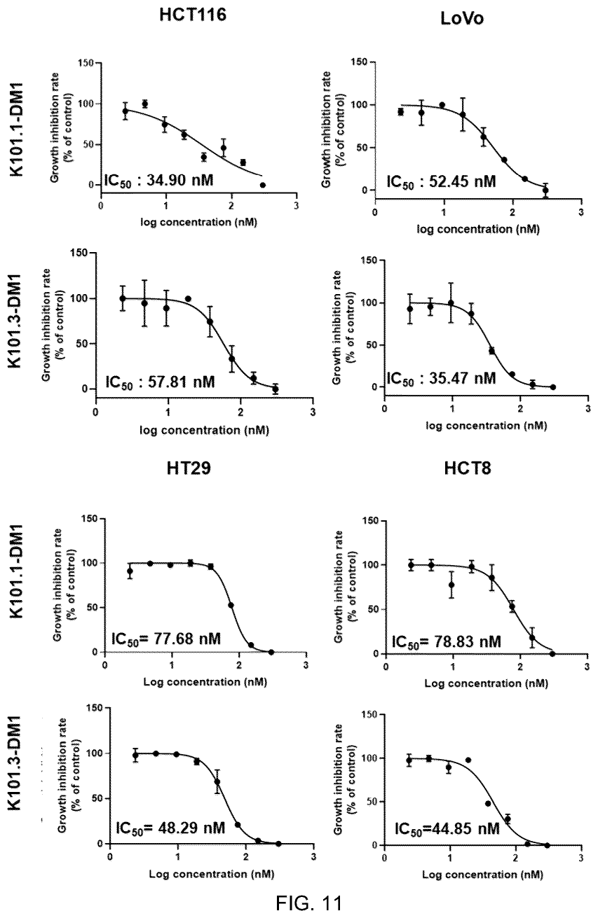

The results are shown in Table 1 and FIG. 11.

As presented in Table 1 and FIG. 11, the IC₅₀ values of K101.1-DM1 in CRC cells ranged from approximately 34.9 to 78.83 nM, while the IC₅₀ values of K101.3-DM1 ranged from approximately 35.47 to 57.81 nM. These findings indicate that GRP94 ADCs specifically inhibit CRC cell growth (Table 1, FIG. 11).

TABLE 1

| Classification | HCT116 | LoVo | HT29 | HCT8 |
|---|---|---|---|---|
| K101.1-DM1 | 34.90 nM | 52.45 nM | 77.68 nM | 78.83 nM |
| K101.3-DM1 | 57.81 nM | 35.47 nM | 48.29 nM | 44.85 nM |

### Example 2-3: Evaluation of the Antitumor Activity of GRP94 Antibody-Drug Conjugates on CRC Tumor Spheroid Formation

Tumor spheroid formation assays are traditionally performed on three-dimensional tumor spheroids of cancer cells to evaluate the in vivo antiproliferative or cytotoxic effects of drugs. First, 1×10³ CRC (HCT116, HCT8, HT29, or LoVo) cells were seeded in 96-well ultralow attachment round-bottom microplates to form spheroids. To evaluate the effects of K101.1-DM1 or K101.3-DM1 on HCT116 spheroids, K101.1 and K101.3 were treated at 150 nM, while K101.1-DM1 and K101.3-DM1 were treated at 50 nM or 150 nM.

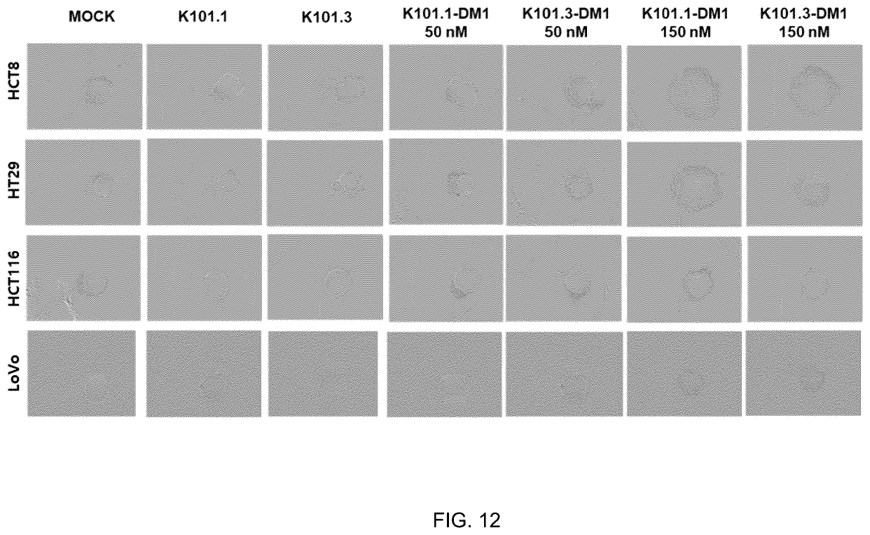

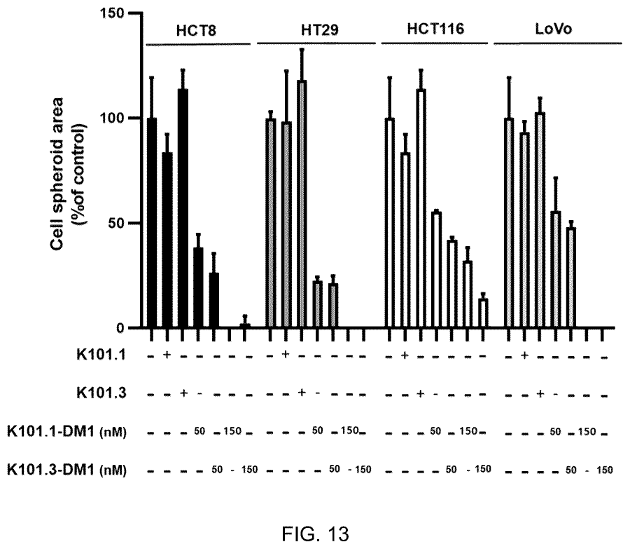

The results are shown in FIGS. 12 and 13.

As shown in FIGS. 12 and 13, K101.1-DM1 and K101.3-DM1 significantly inhibited spheroid formation by approximately 50% to 100% compared to untreated CRC cells. These results demonstrate that K101.1-DM1 and K101.3-DM1 mediate a strong inhibitory effect on CRC spheroid formation.

The present disclosure has been described in detail with reference to specific embodiments. However, it is evident that such specific descriptions merely represent preferred embodiments and do not limit the scope of the present disclosure to those examples.

1. An antibody-drug conjugate (ADC) comprising an antibody or an antigen-binding fragment thereof that specifically binds to GRP94, and a drug, wherein the antibody or antigen-binding fragment thereof comprises:
(i) a heavy chain complementarity-determining region 1 (HCDR1) comprising the amino acid sequence of SEQ ID NO: 1, a heavy chain complementarity-determining region 2 (HCDR2) comprising the amino acid sequence of SEQ ID NO: 2, a heavy chain complementarity-determining region 3 (HCDR3) comprising the amino acid sequence of SEQ ID NO: 3, a light chain complementarity-determining region 1 (LCDR1) comprising the amino acid sequence of SEQ ID NO: 4, a light chain complementarity-determining region 2 (LCDR2) comprising the amino acid sequence of SEQ ID NO: 5, and a light chain complementarity-determining region 3 (LCDR3) comprising the amino acid sequence of SEQ ID NO: 6; or
(ii) a HCDR1 comprising the amino acid sequence of SEQ ID NO: 9, a HCDR2 comprising the amino acid sequence of SEQ ID NO: 10, a HCDR3 comprising the amino acid sequence of SEQ ID NO: 11, a LCDR1 comprising the amino acid sequence of SEQ ID NO: 12, a LCDR2 comprising the amino acid sequence of SEQ ID NO: 13, and a LCDR3 comprising the amino acid sequence of SEQ ID NO: 14.

2. The antibody-drug conjugate of claim 1, wherein the antibody or antigen-binding fragment thereof of (i) comprises a heavy chain variable region comprising the amino acid sequence of SEQ ID NO: 7 and a light chain variable region comprising the amino acid sequence of SEQ ID NO: 8.

3. The antibody-drug conjugate of claim 1, wherein the antibody or antigen-binding fragment thereof of (ii) comprises a heavy chain variable region comprising the amino acid sequence of SEQ ID NO: 15 and a light chain variable region comprising the amino acid sequence of SEQ ID NO: 16.

4. The antibody-drug conjugate of claim 1, wherein the antibody or antigen-binding fragment thereof in (i) or (ii) binds to a GRP94 target antigen expressed on the cell membrane or within the cell membrane of cancer cells, and the antibody, after binding to the GRP94 target antigen, is internalized into the cell.

5. The antibody-drug conjugate of claim 1, wherein the antibody or antigen-binding fragment thereof comprises selenocysteine in the constant region.

6. The antibody-drug conjugate of claim 5, wherein the drug is conjugated to the selenocysteine.

7. The antibody-drug conjugate of claim 1, wherein the drug is conjugated to a lysine residue of the antibody.

8. The antibody-drug conjugate of claim 1, further comprising a linker that connects the antibody or antigen-binding fragment thereof and the drug.

9. The antibody-drug conjugate of claim 1, wherein the drug is a maytansinoid.

10. The antibody-drug conjugate of claim 1, comprising 1 to 20 units of the drug per antibody or antigen-binding fragment thereof.

11. A pharmaceutical composition for treating cancer, inhibiting cancer metastasis, or inhibiting angiogenesis, comprising the antibody-drug conjugate of claim 1.

12. The pharmaceutical composition of claim 11, wherein the cancer comprises one or more cells that express GRP94 at elevated levels compared to non-cancerous cells.

13. A method for treating cancer, inhibiting cancer metastasis, or inhibiting angiogenesis, the method comprising:
administering to a subject in need thereof a pharmaceutical composition comprising the antibody-drug conjugate of claim 1.
